(19) United States
(12) Patent Application Publication
(10) Pub. No.: US 2023/0024998 A1
(43) Pub. Date: Jan. 26, 2023

(54) SYSTEMS AND METHODS FOR REDUCING INTERFERENCE BETWEEN MRI APPARATUS AND ULTRASOUND SYSTEMS

(71) Applicant: INSIGHTEC, LTD., Tirat Carmel (IL)

(72) Inventor: Adi GREENBERG, Haifa Ot (IL)

(21) Appl. No.: 17/778,364

(22) PCT Filed: Dec. 11, 2020
(86) PCT No.: PCT/IB2020/001032

§ 371 Date: May 19, 2022

Related U.S. Application Data
(60) Provisional application No. 62/947,234, filed on Dec. 12, 2019.

(51) Int. Cl. G01R 33/48; A61B 8/00; G01S 15/89

(52) U.S. Cl. CPC G01R 33/4814; A61B 8/4416; A61B 8/4488; G01S 15/8915; A61N 7/00

(57) ABSTRACT

Approaches for performing magnetic resonance (MR) imaging of an anatomic region in conjunction with an ultrasound operation on the anatomic region include transmitting multiple ultrasound waves or pulses having a fundamental frequency and multiple harmonics to the anatomic region; transmitting an MR pulse sequence to the anatomic region and receiving, therefrom, MR signals within a band of frequencies; and causing the band of frequencies to be located between two adjacent frequencies of the harmonics.

### RELATED APPLICATION

This application claims the benefit of and priority to U.S. Provisional Patent Application No. 62/947,234, filed on Dec. 12, 2019, the entire disclosure of which is hereby incorporated by reference in its entirety.

### FIELD OF THE INVENTION

The present invention relates, generally, to medical diagnosis and treatment guided by magnetic resonance imaging (MRI), and, more specifically, to approaches for reducing interference between the MRI apparatus and ultrasound systems for medical diagnosis and treatment.

### BACKGROUND

Magnetic resonance imaging may be used in conjunction with ultrasound focusing in a variety of medical applications. Ultrasound penetrates well through soft tissues and, due to its short wavelengths, can be focused to spots with dimensions of a few millimeters. As a consequence of these properties, ultrasound can be and has been used for various diagnostic and therapeutic medical purposes, including ultrasound imaging and non-invasive surgery. For example, focused ultrasound may be used to ablate diseased (e.g., cancerous) tissue without causing significant damage to surrounding healthy tissue. An ultrasound focusing system generally utilizes an acoustic transducer surface, or an array of transducer surfaces, to generate an ultrasound beam. In transducer arrays, the individual surfaces, or “elements,” are typically individually controllable—i.e., their vibration phases and/or amplitudes can be set independently of one another—allowing the beam to be steered electronically in a desired direction and focused at a desired distance. The ultrasound system often also includes receiving elements, integrated into the transducer array or provided in form of a separate detector, that help monitor the focused ultrasound treatment, primarily for safety purposes. For example, the receiving elements may serve to detect ultrasound reflected off interfaces between the transducer and the target tissue, which may result from air bubbles on the skin that need to be removed to avoid skin burns. The receiving elements may also be used to detect cavitation in overheated tissues (i.e., the formation of cavities due to the collapse of bubbles formed in the liquid of the tissue).

To visualize the target tissue and guide the ultrasound focus during therapy, MRI may be used. In brief, MRI involves placing a subject, such as the patient, into a homogeneous static magnetic field, thus aligning the spins of hydrogen nuclei in the tissue. Then, by applying a radio-frequency (RF) electromagnetic pulse of the right frequency (the “resonance frequency”), the spins may be flipped, temporarily destroying the alignment and inducing a response signal. Different tissues produce different response signals, resulting in a contrast among these tissues in MR images. Because the resonance frequency and the frequency of the response signal depend on the magnetic field strength, the origin and frequency of the response signal can be controlled by superposing magnetic gradient fields onto the homogeneous field to render the field strength dependent on position. By using time-variable gradient fields, MRI “scans” of the tissue can be obtained. Many MRI protocols utilize time-dependent gradients in two or three mutually perpendicular directions. The relative strengths and timing of the gradient fields and RF pulses are specified in a pulse sequence and may be illustrated in a pulse sequence diagram.

Time-dependent magnetic field gradients may be exploited, in combination with the tissue dependence of the MRI response signal, to visualize, for example, a brain tumor, and determine its location relative to the patient's skull. An ultrasound transducer system, such as an array of transducers attached to a housing, may then be placed on the patient's head. The ultrasound transducer may include MR tracking coils or other markers for determining its position and orientation relative to the target tissue in the MR image. Based on computations of the required transducer element phases and amplitudes, the transducer array is then driven so as to focus ultrasound into the tumor. Alternatively or additionally, the ultrasound focus itself may be visualized, using a technique such as thermal MRI or acoustic resonance force imaging (ARFI), and the measured focus location may be used to adjust the beam orientation. These methods are generally referred to as MR-guided focusing of ultrasound (MRgFUS).

In addition, an MRI apparatus and an ultrasound imaging system may be combined to offer the strengths of both imaging modalities and thereby provide novel insights into the morphology and function of normal and diseased tissues. MRI is used widely for both diagnostic and therapeutic applications because of its multi-planar imaging capability, high signal-to-noise ratio, and sensitivity to subtle changes in soft tissue morphology and function. Ultrasound imaging, on the other hand, has advantages including high temporal resolution, high sensitivity to acoustic scatters (such as calcifications and gas bubbles), excellent visualization, and measurement of blood flow, low cost, and portability. Combining these complementary modalities has provided benefits in intraoperative neurosurgical applications and breast biopsy guidance. By performing imaging with both modalities simultaneously, complications such as spatial and temporal registration between data sets may be simplified. In addition, measurements of unique physiological parameters can be made with each modality to fully characterize the organ or tissue under evolution.

The simultaneous operation of ultrasound and MRI apparatus, however, can lead to undesired interferences. For example, MRI is very sensitive to RF noise generated by the focused ultrasound system (see, e.g., U.S. Pat. No. 6,735,461). Conversely, focused ultrasound procedures often involve RF-sensitive operations (such as the ultrasound detection that may accompany treatment with focused ultrasound) that are easily disturbed by RF excitation signals and/or time-varying field gradients generated by the MRI system. Prior-art approaches to avoiding such interference typically involve use of linear ultrasound amplifiers and high-frequency signal filters; these approaches, however, consume space and power.

Accordingly, there is a need for alternative approaches in MRgFUS applications to minimize or avoid interferences between ultrasound and MR systems.

### SUMMARY

Embodiments of the present invention provide various approaches to concurrently operating an MRI apparatus for imaging an anatomic region and an ultrasound system for diagnostic and/or therapeutic purposes without, or with reduced, interference therebetween. In various embodiments, the ultrasound system and/or MRI apparatus are configured to have low-phase-noise specifications so as to generate localized (e.g., with low phase noises) ultrasound frequencies. For example, the ultrasound system may employ a frequency generator and/or switch elements (e.g., a switching amplifier) that have low jitter for reducing the phase noise associated with the fundamental frequency and the harmonics generated by the ultrasound system. Additionally or alternatively, the low-jitter frequency generator implemented in the ultrasound system (and, in some embodiments, the MRI apparatus) may employ an oscillator having a low frequency drift for increasing the stability of the generated frequencies. In one embodiment, the oscillator includes a phase-locked loop (PLL) and/or a direct-digital-synthesis (DDS) circuit to lock the time (and thereby the phase) of the generated signals to the time (and thereby the phase) of an internal clock of the MRI apparatus for further improving the stability of the generated frequencies. These approaches may effectively ensure that the operation frequencies of the ultrasound system and the MRI apparatus—and thereby the received MRI signals—are stable (e.g., having low drifts and thereby being temporarily “locked”) and localized (e.g., have low phase noises).

In various embodiments, after the signals generated by the ultrasound system and/or MRI apparatus are localized and stable, the fundamental frequency generated by the ultrasound system may be adjusted such that the band of the received MR signals falls between the peaks of two adjacent harmonics to ensure minimal interference therebetween. Thereafter, the interference caused by the ultrasound system in the received MR signals may be filtered or subtracted utilizing a suitable conventional technique.

In some embodiments, the MRI apparatus is idling (i.e., inactive or not actively transmitting any MR pulses to the target but capable of detecting signals) while the ultrasound system actively transmits waves. The detected MRI signals resulting from operation of the ultrasound system while the MRI apparatus is idling may serve as reference (or baseline) signals for correcting the received MR signals measured when both the MRI apparatus and ultrasound system are operated concurrently. For example, the received MR signals measured when both the MRI apparatus and ultrasound system are active may be corrected by subtracting the previously obtained reference signals therefrom.

In various embodiments, the RF transmission pulses in an MR pulse sequence are configured to have alternating phases between two consecutive repetitions. This may advantageously allow the interference between signals generated by the ultrasound system and the MRI apparatus to be “aliased” (or shifted) outside the k-space spectrum of the received MR signals. Additionally or alternatively, the bandwidth of the received MR signals may be narrowed by, for example, increasing the MR sampling time and/or reducing the number of measured MR samples so as to reduce interference with the ultrasound system. In one embodiment, the fundamental frequency of signals generated by the ultrasound system is adjusted such that the harmonic(s) associated therewith fall in location(s) within the frequency band of the MR received signals that are less important for constructing the MR images.

In various embodiments, the ultrasound system operates on a pulsed basis. To avoid (or at least reduce) the interference between the ultrasound system and the MRI apparatus, the waveform of the ultrasound pulses may be shaped such that the resulting fundamental frequency and harmonics form narrow bands and can thereby be easily filtered or subtracted from the received MR signals. Additionally or alternatively, the ultrasound pulses may be regulated such that the phase and/or time delay between at least some adjacent pulses are different (or, in one embodiment, random). As a result, the noise associated the pulses will be stochastically spread over the spectrum and will thus average out; this approach may effectively reduce the interference noise in the received MR signals.

Accordingly, in one aspect, the invention pertains to a system for performing magnetic resonance (MR) imaging of an anatomic region in conjunction with an ultrasound operation on the anatomic region. In various embodiments, the system includes an MR imaging apparatus for imaging the anatomic region; an ultrasound transducer system for performing the ultrasound operation; and a controller in communication with the MR imaging apparatus and ultrasound transducer system. In one implementation, the controller is configured to cause the ultrasound transducer system to transmit, to the anatomic region, ultrasound waves or pulses having a fundamental frequency and multiple harmonics; cause the MR imaging apparatus to transmit an MR pulse sequence to the anatomic region and receive, therefrom, MR signals within a band of frequencies; and cause the band of the frequencies to be located between two adjacent frequencies of the harmonics.

In some embodiments, the ultrasound transducer system includes a low-jitter frequency generator and/or a low-jitter switch element for reducing a phase noise associated with the fundamental frequency and harmonics. In addition, the ultrasound transducer system and/or the MR imaging apparatus may include one or more oscillators having a low frequency drift so as to improve stability of the fundamental frequency, the harmonics and/or a frequency associated with ultrasound waves or pulses transmitted by the MR imaging apparatus. The oscillator(s) may include a phase-locked loop for locking the phase associated with the fundamental frequency, the harmonics and/or the frequency associated with the ultrasound waves or pulses transmitted by the MR imaging apparatus to an internal clock of the MR imaging apparatus.

In some embodiments, the controller is further configured to filter or subtract the fundamental frequency and harmonics from the received MR signals. In addition, the fundamental frequency may be larger than a bandwidth of the received MR signals. In one embodiment, the MR pulse sequence includes RF transmission pulses having alternating phases between two consecutive repetitions. The controller may be further configured to cause the MR imaging apparatus to detect reference MR signals in response to transmission of the ultrasound waves or pulses thereto prior to causing the MR imaging apparatus to transmit the MR pulse sequence to the anatomic region; and adjust the received MR signals based at least in part on the reference MR signals.

In various embodiments, the controller is further configured to reduce a bandwidth of the received MR signals. In addition, the controller may be further configured to increase an MR scanning time or reduce a number of measured MR signals. In one implementation, the controller is further configured to shape a waveform of one or more of the ultrasound pulses. In addition, the controller may be further configured to implement a Gaussian filter, a raised-cosine filter, and/or a sinc filter for shaping the waveform of the ultrasound pulse(s). The controller may be further configured to regulate the ultrasound pulses such that a phase and/or a time delay between some of the pulses are different. In one embodiment, the controller is implemented in the ultrasound transducer system.

In another aspect, the invention relates to a method of performing magnetic resonance (MR) imaging of an anatomic region in conjunction with an ultrasound operation on the anatomic region. In various embodiments, the method includes transmitting multiple ultrasound waves or pulses having a fundamental frequency and multiple harmonics to the anatomic region; transmitting an MR pulse sequence to the anatomic region and receiving, therefrom, MR signals within a band of frequencies; and causing the band of frequencies to be located between two adjacent frequencies of the harmonics.

The method may further include filtering or subtracting the fundamental frequency and harmonics from the received MR signals. The fundamental frequency may be larger than a bandwidth of the received MR signals. In addition, the MR pulse sequence may include RF transmission pulses having alternating phases between two consecutive repetitions. In some embodiments, the method further includes causing the MR imaging apparatus to detect reference MR signals in response to transmission of the ultrasound waves or pulses thereto prior to causing the MR imaging apparatus to transmit the MR pulse sequence to the anatomic region; and adjusting the received MR signals based at least in part on the reference MR signals.

Additionally, the method may further include reducing a bandwidth of the received MR signals. In one embodiment, the method further includes increasing an MR scanning time or reducing a number of measured MR signals. In addition, the method may further include shaping a waveform of one or more of the ultrasound pulses. For example, the waveform of the ultrasound pulse(s) may be shaped by a Gaussian filter, a raised-cosine filter, and/or a sinc filter. In one embodiment, the method further includes regulating the ultrasound pulses such that a phase and/or a time delay between some of the pulses are different.

Another aspect of the invention relates to a system for performing magnetic resonance (MR) imaging of an anatomic region in conjunction with an ultrasound operation on the anatomic region. In various embodiments, the system includes an MR imaging apparatus for imaging the anatomic region; an ultrasound transducer system for performing the ultrasound operation; and a controller in communication with the MR imaging apparatus and ultrasound transducer system. In one implementation, the controller is configured to cause the ultrasound transducer system to transmit, to the anatomic region, ultrasound waves or pulses having a fundamental frequency and multiple harmonics; and cause the MR imaging apparatus to transmit an MR pulse sequence having multiple RF transmission pulses to the anatomic region and receive, therefrom, MR signals within a band of frequencies. In addition, the RF transmission pulses may have alternating phases between two consecutive repetitions.

The ultrasound transducer system may include a low-jitter frequency generator and/or a low-jitter switch element for reducing a phase noise associated with the fundamental frequency and harmonics. In addition, the ultrasound transducer system or the MR imaging apparatus comprises at least one oscillator having a low frequency drift so as to improve stability of the fundamental frequency, the harmonics and/or a frequency associated with ultrasound waves or pulses transmitted by the MR imaging apparatus. The oscillator(s) may include a phase-locked loop for locking the phase associated with the fundamental frequency, the harmonics and/or the frequency associated with the ultrasound waves or pulses transmitted by the MR imaging apparatus to an internal clock of the MR imaging apparatus.

In some embodiments, the controller is further configured to filter or subtract the fundamental frequency and harmonics from the received MR signals. In addition, the fundamental frequency is smaller than a bandwidth of the received MR signals. The controller may be further configured to cause the MR imaging apparatus to detect reference MR signals in response to transmission of the ultrasound waves or pulses thereto prior to causing the MR imaging apparatus to transmit the MR pulse sequence to the anatomic region; and adjust the received MR signals based at least in part on the reference MR signals.

In various embodiments, the controller is further configured to reduce a bandwidth of the received MR signals. In addition, the controller may be further configured to increase an MR scanning time or reduce a number of measured MR signals. In one embodiment, the controller is further configured to shape a waveform of one or more of the ultrasound pulses. For example, the controller may be configured to implement a Gaussian filter, a raised-cosine filter, and/or a sinc filter for shaping the waveform of the ultrasound pulse(s). Additionally, the controller may be further configured to regulate the ultrasound pulses such that a phase and/or a time delay between some of the pulses are different. In one embodiment, the controller is implemented in the ultrasound transducer system.

In yet another aspect, the invention pertains to a method of performing magnetic resonance (MR) imaging of an anatomic region in conjunction with an ultrasound operation on the anatomic region. In various embodiments, the method includes transmitting multiple ultrasound waves or pulses having a fundamental frequency and multiple harmonics to the anatomic region; and transmitting an MR pulse sequence having multiple RF transmission pulses to the anatomic region and receiving, therefrom, MR signals within a band of frequencies. In one implementation, the RF transmission pulses have alternating phases between two consecutive repetitions.

The method may further include filtering or subtracting the fundamental frequency and harmonics from the received MR signals. In addition, the fundamental frequency is smaller than a bandwidth of the received MR signals. In some embodiments, the method further includes causing the MR imaging apparatus to detect reference MR signals in response to transmission of the ultrasound waves or pulses thereto prior to causing the MR imaging apparatus to transmit the MR pulse sequence to the anatomic region; and adjusting the received MR signals based at least in part on the reference MR signals.

Additionally, the method may further include reducing a bandwidth of the received MR signals. In some embodiments, the method further include increasing an MR scanning time or reducing a number of measured MR signals. In addition, the method may further include shaping a waveform of one or more of the ultrasound pulses. For example, the waveform of the ultrasound pulse(s) may be shaped by a Gaussian filter, a raised-cosine filter, and/or a sinc filter. In one embodiment, the method further includes regulating the ultrasound pulses such that a phase and/or a time delay between some of the pulses are different. In one embodiment, the method further includes regulating the ultrasound pulses such that a phase and/or a time delay between some of the pulses are different.

As used herein, the term “substantially” means±10%, and in some embodiments, ±5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which:

FIG. 1A schematically depicts an exemplary MRI system in accordance with various embodiments of the current invention;

FIG. 1B schematically depicts an exemplary ultrasound system in accordance with various embodiments of the current invention;

FIG. 2 schematically illustrates an interaction between an MRI system and an ultrasound transducer system in accordance with various embodiments of the present invention;

FIGS. 3A and 3C schematically depict frequencies generated by an ultrasound system and a frequency band associated with the received MR signals in accordance with various embodiments of the present invention;

FIG. 3B depicts a phase noise component associated with an oscillator's carrier frequency in accordance with various embodiments of the present invention;

FIG. 4 illustrates exemplary MR pulse sequences and received MR echo signals in accordance with various embodiments of the present invention;

FIG. 5 depicts signals detected by an MRI apparatus in accordance with various embodiments of the present invention;

FIG. 6A depicts concurrent operations of an ultrasound system and an MRI apparatus in accordance with various embodiments of the present invention;

FIG. 6B schematically depicts a shaped ultrasound pulse in accordance with various embodiments of the present invention;

FIG. 6C schematically depicts an ultrasound pulse train in accordance with various embodiments of the present invention;

FIG. 6D schematically depicts an ultrasound pulse train having shaped pulses in accordance with various embodiments of the present invention; and

FIGS. 7A and 7B are flow charts illustrating approaches for eliminating/reducing interference between an ultrasound system and an MRI apparatus in accordance with various embodiments of the present invention.

### DETAILED DESCRIPTION

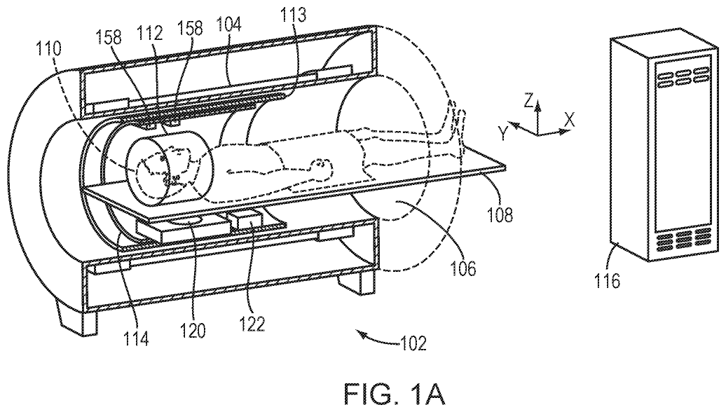

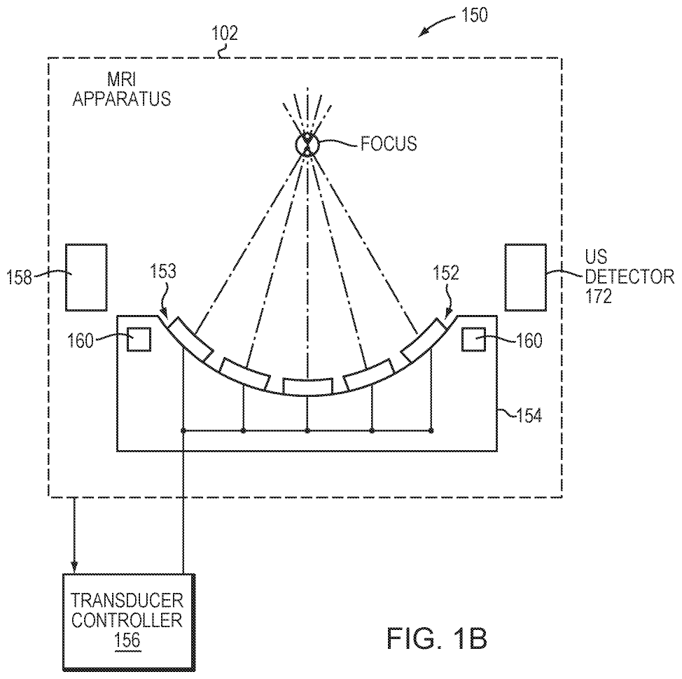

FIG. 1A illustrates an exemplary MRI apparatus 102. The apparatus 102 may include a cylindrical electromagnet 104, which generates the requisite static magnetic field within a bore 106 of the electromagnet 104. During medical procedures, a patient is placed inside the bore 106 on a movable support table 108. A region of interest 110 within the patient (e.g., the patient's head) may be positioned within an imaging region 112 wherein the electromagnet 104 generates a substantially homogeneous field. A set of cylindrical magnet field gradient coils 113 may also be provided within the bore 106 and surrounding the patient. The gradient coils 113 generate magnetic field gradients of predetermined magnitudes, at predetermined times, and in three mutually orthogonal directions. With the field gradients, different spatial locations can be associated with different precession frequencies, thereby giving an MR image its spatial resolution. An RF transmitter coil 114 surrounding the imaging region 112 emits RF pulses into the imaging region 112 to cause the patient's tissues to emit magnetic-resonance (MR) response signals. Raw MR response signals are sensed by the RF coil 114 and passed to an MR controller 116 that then computes an MR image, which may be displayed to the user. Alternatively, separate MR transmitter and receiver coils may be used. Images acquired using the MRI apparatus 102 may provide radiologists and physicians with a visual contrast between different tissues and detailed internal views of a patient's anatomy that cannot be visualized with conventional x-ray technology.

The MRI controller 116 may control the pulse sequence, i.e., the relative timing and strengths of the magnetic field gradients and the RF excitation pulses and response detection periods. The MR response signals are amplified, conditioned, and digitized into raw data using an image processing system, and further transformed into arrays of image data by methods known to those of ordinary skill in the art. Based on the image data, a treatment region (e.g., a tumor) is identified. The image processing system may be part of the MRI controller 116, or may be a separate device (e.g., a general-purpose computer containing image processing software) in communication with the MRI controller 116. In some embodiments, one or more ultrasound systems 120 or one or more sensors 122 are displaced within the bore 106 of the MRI apparatus 102 as further described below.

FIG. 1B illustrates an exemplary system 150, such as an ultrasound system, concurrently operated with the MRI system 102 in accordance with some embodiments of the present invention, although alternative concurrently operated systems with ultrasound or other functionality that may interfere with the MRI system 102 are also within the scope of the invention. As shown, the ultrasound system includes a plurality of ultrasound transducer elements 152, which are arranged in an array 153 at the surface of a housing 154. The array may comprise a single row or a matrix of transducer elements 152. In alternative embodiments, the transducer elements 152 may be arranged without coordination, i.e., they need not be spaced regularly or arranged in a regular pattern. The array may have a curved (e.g., spherical or parabolic) shape, as illustrated, or may include one or more planar or otherwise shaped sections. Its dimensions may vary, depending on the application, between millimeters and tens of centimeters. The transducer elements 152 may be piezoelectric ceramic elements. Piezo-composite materials, or generally any materials capable of converting electrical energy to acoustic energy, may also be used. To damp the mechanical coupling between the elements 152, they may be mounted on the housing 154 using silicone rubber or any other suitable damping material.

The transducer elements 152 are separately controllable, i.e., they are each capable of emitting ultrasound waves at amplitudes and/or phases that are independent of the amplitudes and/or phases of the other transducers. A transducer controller 156 serves to drive the transducer elements 152. For n transducer elements, the controller 156 may contain n control circuits each comprising an amplifier and a phase delay circuit, each control circuit driving one of the transducer elements. The controller 156 may split an RF input signal, typically in the range from 0.1 MHz to 10 MHz, into n channels for the n control circuit. It may be configured to drive the individual transducer elements 152 of the array at the same frequency, but at different phases and different amplitudes so that they collectively produce a focused ultrasound beam. In some embodiments, each transducer element 152 is connected to the same or a different signal driver via a corresponding channel and a corresponding switch element in a switch matrix. By toggling the switches in the switch matrix, their corresponding transducer elements may be activated and deactivated. The transducer controller 156 desirably provides computational functionality, which may be implemented in software, hardware, firmware, hardwiring, or any combination thereof, to compute the required phases and amplitudes for a desired focus location. In general, the controller 156 may include several separable apparatus, such as a frequency generator (including an oscillator), a beamformer containing the amplifier and phase delay circuitry, and a computer (e.g., a general-purpose computer) performing the computations and communicating the phases and amplitudes for the individual transducer elements 152 to the beamformer. Such systems are readily available or can be implemented without undue experimentation.

To perform ultrasound imaging, the controller 156 drives the transducer elements 152 to transmit acoustic signals into a region being imaged and to receive reflected signals from various structures and organs within the patient's body. By appropriately delaying the pulses applied to each transducer element 152, a focused ultrasound beam can be transmitted along a desired scan line. Acoustic signals reflected from a given point within the patient's body are received by the transducer elements 152 at different times. The transducer elements can then convert the received acoustic signals to electrical signals which are supplied to the beamformer. The delayed signals from each transducer element 152 are summed by the beamformer to provide a scanner signal that is a representation of the reflected energy level along a given scan line. This process is repeated for multiple scan lines to provide signals for generating an image of the prescribed region of the patient's body. Typically, the scan pattern is a sector scan, wherein the scan lines originate at the center of the ultrasound transducer and are directed at different angles. A linear, curvilinear or any other scan pattern can also be utilized.

The ultrasound system may be disposed within the bore 106 of the MRI apparatus 102 or placed in the vicinity of the MRI apparatus 102. To aid in determining the relative positions of the ultrasound system 150 and MRI apparatus 102, the ultrasound system 150 may further include MR trackers 160 associated therewith, arranged at a fixed position and orientation relative to the system 150. The trackers 160 may, for example, be incorporated into or attached to the ultrasound system housing. If the relative positions and orientations of the MR trackers 160 and ultrasound system 150 are known, MR scans of the MR trackers 160 implicitly reveal the location of the ultrasound system 150 in MRI coordinates, i.e., in the coordinate system of the MRI apparatus 102.

As depicted in FIGS. 1A and 1B, a combined system including the MRI apparatus 102 and ultrasound system 150 may be capable of imaging the anatomic region of interest and detecting ultrasound signals; the combined system may serve to monitor the application of ultrasound for treatment and/or safety purposes. For example, ultrasound reflections off tissue interfaces along the ultrasound beam path may be analyzed to ensure, if necessary by adjustment of the treatment protocol, that such interfaces are not inadvertently overheated. Further, measurements of the received cavitation spectrum may be used to detect cavitation resulting from the interaction of ultrasound energy with water-containing tissue. In addition, the visualization of the tissue and target may be supplemented by ultrasound imaging, for example, to facilitate tracking a moving target. Ultrasound detection may be accomplished with the ultrasound transducer array 153. For example, treatment and imaging periods may be interleaved, or a contiguous portion of the array 153 or discontiguous subset of transducer elements 152 may be dedicated to imaging while the remainder of the array 153 focuses ultrasound for treatment purposes. Alternatively, a separate ultrasound receiver 172—e.g., a simple ultrasound probe or array of elements—may be provided. The separate receiver 172 may be placed in the vicinity of the ultrasound transducer array 153, or integrated into its housing 154. In addition, the receiver 172 may be disposed within the bore 106 of the MRI apparatus 102 or placed in the vicinity thereof.

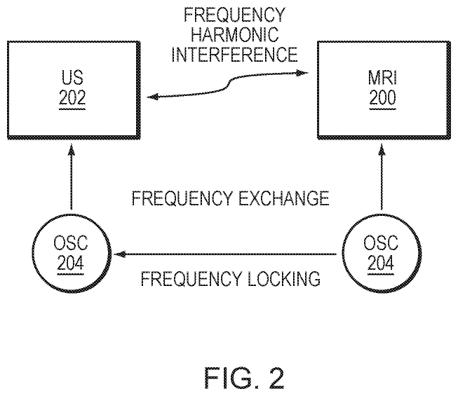

FIG. 2 schematically illustrates the interaction between an MRI apparatus 200 and a phased-array ultrasound transducer system 202 in accordance with various embodiments of the invention. As described above, the MRI apparatus 200 includes a cylindrical electromagnet to generate the requisite static magnetic field, Bo, and RF transmitter coils and gradient coils for generating time-varying magnetic gradients across the tissue to be imaged. Typically, the MRI pulses have frequencies in the range from about 50 MHz to about 150 MHz, and the fundamental operation frequency of the ultrasonic treatment/imaging procedures and/or cavitation detection (or other concurrently performed RF-sensitive operations) ranges from 0.1 MHz to 10 MHz. Thus, the harmonics of the fundamental frequency associated with the ultrasound operation can potentially interfere with the received MR signals. Because the MRI pulse frequency is generally tightly coupled to the applied static magnetic field Bo, various embodiments herein avoid (or at least reduce) the interference between the ultrasound system 202 and the MRI apparatus 200 by causing the fundamental frequency and corresponding harmonics generated by the ultrasound system 202 to be outside the band of the received MR signals as further described below.

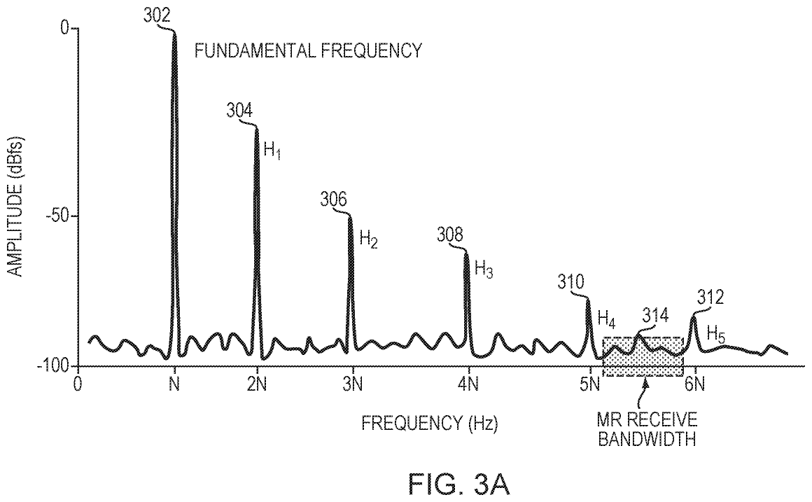

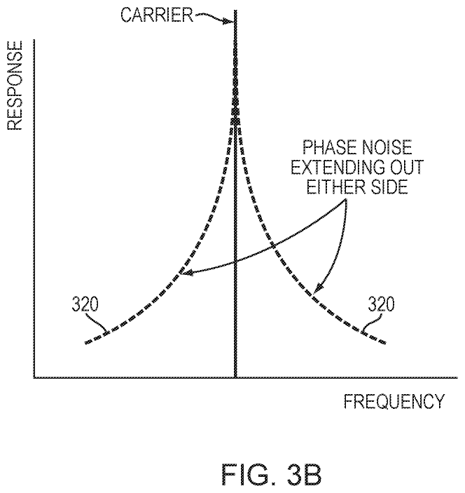

FIG. 3A illustrates a fundamental frequency 302 and its corresponding harmonics 304-312 generated by the ultrasound system 202 for a diagnostic or therapeutic application in accordance herewith. In addition, FIG. 3A schematically depicts frequencies of the received MR signals within a frequency band 314 having a bandwidth that may interfere with the frequencies 302-312 generated by the ultrasound system 202. An ideal oscillator would generate a pure sine wave, which in the frequency domain would be represented as a Dirac delta function at the oscillator's carrier frequency, but a real oscillator typically has phase-modulated noise components. For example, as depicted in FIG. 3B, the phase noise components may spread the power of a signal to adjacent frequencies, resulting in noise sidebands 320. The noise sidebands 320 may sometimes be sufficient to cause interference between the frequencies 302-312 and the received MR signals within the MR band 314. Thus, to eliminate (or at least reduce) the interference, it is critical that the generated frequencies 302-314 are localized (e.g., have a low phase noise or narrow sidebands 320).

In various embodiments, the ultrasound system 202 is configured to have low-phase-noise specifications so as to reduce the phase noise associated with the generated fundamental frequency and corresponding harmonics. For example, the ultrasound system 202 may employ a low-jitter (e.g., having a low phase noise) frequency generator and/or low-jitter switch elements (e.g., a switching amplifier). In one embodiment, the jitter performance of the frequency generator and/or switch elements is less than 1 ps. Additionally or alternatively, the ultrasound system 202 may include a jitter attenuator to reduce the system jitter. In some embodiments, the MRI apparatus 200 also includes a low-jitter frequency generator and/or jitter attenuator to reduce the phase noise associated with its transmission signals.

Additionally or alternatively, referring again to FIG. 2, the oscillator 204 implemented in the ultrasound system 202 and/or MRI apparatus 200 may have a low-frequency drift so as to increase stability of the generated frequencies. For example, the oscillator 204 may have a frequency drift below 1 ppm in a temperature range of −40° C. to 85° C. In some embodiments, the oscillator 204 includes a PLL and/or a DDS circuit to lock the frequency of the ultrasound signals to an MR internal clock of the MRI apparatus 200; this may further improve stability of the generated frequencies. These approaches may effectively ensure that the operational frequencies of the ultrasound system 202 and/or the MRI apparatus 200 (and thereby the frequency band 314 of the received MR signals) are stable (e.g., tied together and thereby being “locked” and having no (or at least very limited) frequency drifts). As a result, the interference caused by the frequencies associated with the ultrasound system 202 and the MR apparatus 200 may also be stable; this thereby allows the interference to be more easily filtered or subtracted from the received MR signals using a conventional filtering/subtracting technique. For example, a median filter or a low-pass filter may be implemented to filter the interference from the received MR signals. Additionally or alternatively, one or more MR reference (or baseline) signals, acquired when the MRI apparatus is idling while the ultrasound system actively transmits, may be utilized to correct the MR signals measured when both the MRI apparatus and ultrasound system are active as further described below.

Referring again to FIG. 3A, in various embodiments, after ensuring that the frequencies 302-314 generated by the ultrasound system 202 and/or the frequency band 314 associated with the received MR signals are localized (e.g., having a low phase noise) and stable (e.g., having a low drift), the fundamental frequency 302 generated by the ultrasound system 202 is adjusted such that the frequency band 314 of the received MR signals falls between the peaks (and their associated phase noise components) of two adjacent harmonics (e.g., harmonics 310, 312 as depicted). Thus, the frequency difference between adjacent harmonics of the generated ultrasound signals is preferably larger than the bandwidth of the frequency band 314 associated with the received MR signals. This can be achieved by, for example, adjusting the fundamental frequency 302 of the ultrasound system 202 such that it is larger than the bandwidth. Referring to FIG. 3C, in one embodiment, the fundamental frequency of the ultrasound system 202 is selected to satisfy the following equations:

N×f_{ultrasound}<f_{MR}−0.5×BW_{MR}, Eq. (1)

(N+1)×f_{ultrasound}>f_{MR}+0.5×BW_{MR}, Eq. (2)

where f_{ultrasound} denotes the fundamental frequency generated by the ultrasound system 202; N and N+1 denote the N^{th} and (N+1)^{th} harmonics, respectively, associated with the fundamental frequency; f_{MR} denotes the central frequency of the received MR signals; and BW_{MR} denotes the bandwidth of the received MR signals. This approach is particularly suitable for MR scans that have a relative narrow bandwidth BW_{MR} of the received signals.

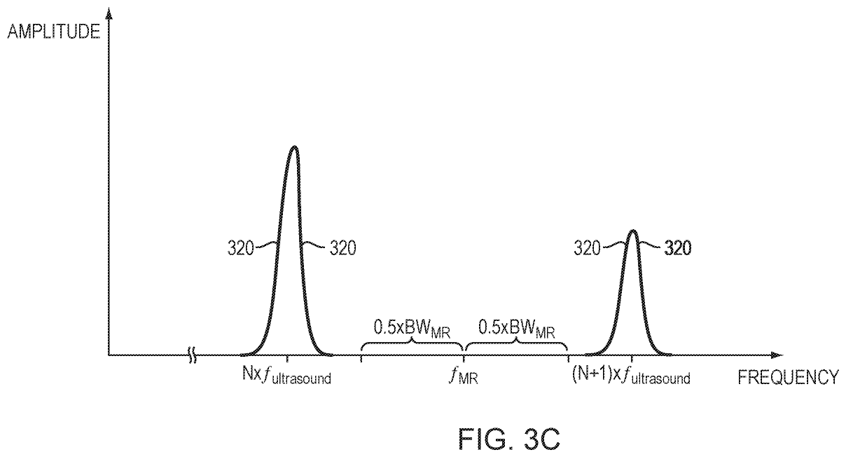

In some embodiments, the fundamental frequency 302 generated by the ultrasound system 202 is smaller than the bandwidth of the received MR signals and the harmonic(s) may be located within the MR band 314; as a result, the fundamental frequency 302 may not satisfy Eqs. (1) and (2) set forth above. This may occur when, for example, the MR scans have a wide bandwidth associated with the received signals and/or the fundamental frequency 302 generated by the ultrasound system 202 is determined based on the requirements of the ultrasound diagnostic and therapeutic application (such as maximizing the peak acoustic intensity and/or optimizing the focusing properties at the target region as described in U.S. Patent Publication Nos. 2016/0008633 and 2020/0205782, the contents of which are incorporated herein by reference). This situation may be acceptable so long as the difference between the determined fundamental frequency and the MR bandwidth is insignificant (e.g., less than 5% or, in some embodiments, less than 10%). To eliminate (or at least reduce) the interference between the signals generated by the ultrasound system 202 and the MRI apparatus 200 when the fundamental frequency 302 associated with the ultrasound system 202 is smaller than the MR received bandwidth 314, various embodiments adjust the phases associated with the MR transmission pulses. For example, referring to FIG. 4, the MR pulse sequences 402 may include RF transmission pulses 404 having alternating phases between two consecutive repetitions—that is, for each RF pulse applied, a reversed (i.e., having a 180° phase difference) RF pulse is applied at the end of the repetition time (TR). This approach may improve the steady-state magnetization, particularly when a short TR (i.e., a high acquisition rate) is preferred. Typically, the received MR signals 406 from the target tissue in response to the reversed RF pulses are inverted by 180° in phase prior to reconstructing images therefrom. But because the phase alternation has no (or at least very limited) effect on the frequency interference between the ultrasound system 202 and the MRI apparatus 200, the interference may be consistent throughout the entire MR pulse sequences 402. By applying the 180° phase inversion to the constant interference and modulating its phase with the rate of the alternating inversion, the interference may be “aliased”—i.e., shifted—outside the k-space spectrum of the received MR signals. For example, the interference may be shifted from fᵢ to fᵢ+fₘ and fᵢ−fₘ, where fᵢ is the interference frequency (e.g., near the MR center frequency) and fₘ is the modulation frequency). In addition, because the RF transmission pulses between two sequences (and thereby two scans) have alternating phases, the phase noise associated with the received MR signals may advantageously cancel out when reconstructing the MR images.

To alias the interference of frequencies associated with the ultrasound system 202 and the MRI apparatus 200, in various embodiments, the frequency interference is adjusted to be near the center frequency of the MRI pulses (e.g., within a few ppm, or in some embodiments, a few hundred ppm). For example, the controller 156 may select the fundamental frequency 302 of the ultrasound system 202 to satisfy the equation:

N×f_{ultrasound}=f_{MR},

where N denotes the N^{th} harmonic and is preferably a low-amplitude, even-numbered harmonic. The low-amplitude harmonic may thereby result in limited effects on the MR images. Further, after aliasing, any residual interference present in the k-space spectrum may be filtered and/or subtracted using a suitable conventional filtering/subtracting technique as described above.

Additionally or alternatively, upon determining that the fundamental frequency 302 generated by the ultrasound system 202 is smaller than the bandwidth associated with the received MR signals, the controller 116 may narrow the MR bandwidth to reduce the interference with the ultrasound system 202. This may be achieved by, for example, increasing the MR sampling time and/or reducing the number of measured MR samples. In another embodiment, the fundamental frequency of the ultrasound system 202 is adjusted such that the harmonics associated therewith fall in locations within the MR band that are less important for constructing the MR images. For example, if the center of the image is more important (e.g., of greater interest) than the edges of the image, the harmonics may be adjusted to appear in locations that are less relevant for constructing the center of the image.

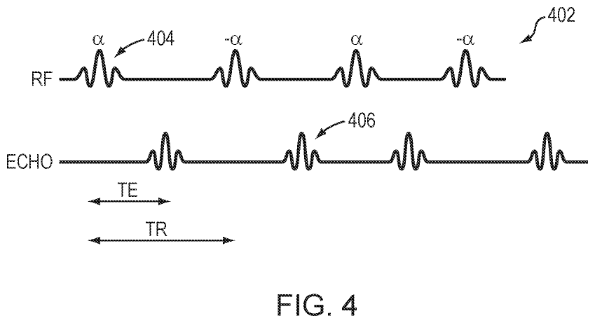

In various embodiments, the interference caused by the harmonics associated with the ultrasound system 202 can be filtered or subtracted from the received MR signals using image processing techniques. Referring to FIG. 5, in various embodiments, prior to activating the MRI apparatus 202 for acquiring images, a k-space or real-space reference (or a baseline) MR image resulting from operation of the ultrasound system 202 can be acquired. For example, the MRI apparatus 200 may be idling—i.e., inactive or not actively transmitting any MR pulses to the target but capable of detecting signals within the band 502—while the ultrasound system 202 actively transmits waves to the target region. The MRI apparatus 202 may then detect one or more signals 504 from the target in its received band 502. The detected signals are referred to herein as reference signals (or baseline signals) that can be further processed to generate the k-space reference image and/or to reconstruct the real-space reference image. During concurrent operation of the MRI apparatus 200 and ultrasound system 202, the MR signals 506-510 from the target may be detected and then corrected by subtracting therefrom the reference signals measured when the MRI apparatus 200 is idling. In one embodiment, the correction is performed at the image level—that is, the k-space or real-space MR image acquired when both MRI apparatus 200 and ultrasound system 202 are operated is corrected by subtracting the k-space or real-space reference image measured when the MRI apparatus 200 is idling therefrom. Approaches for correcting the MR signals 506-510 using the reference signal(s) 504 are provided in, for example, U.S. Pat. No. 10,571,540, the entire disclosure of which is hereby incorporated by reference.

In some embodiments, the controller 116 may average multiple MR signals 506-510 received during concurrent operation of the MRI apparatus 200 and ultrasound system 202 over the spectra, and then identify the stable interference therein based on one or more interference characteristics (e.g., the amplitude, phase, phase drift, etc.). The identified interference can then be filtered and/or subtracted using the conventional technique described above. Additionally or alternatively, conventional machine learning techniques may be implemented to identify the interferences that are periodically observed in the MR images. Again, the identified interference may then be filtered and/or subtracted from the MR images.

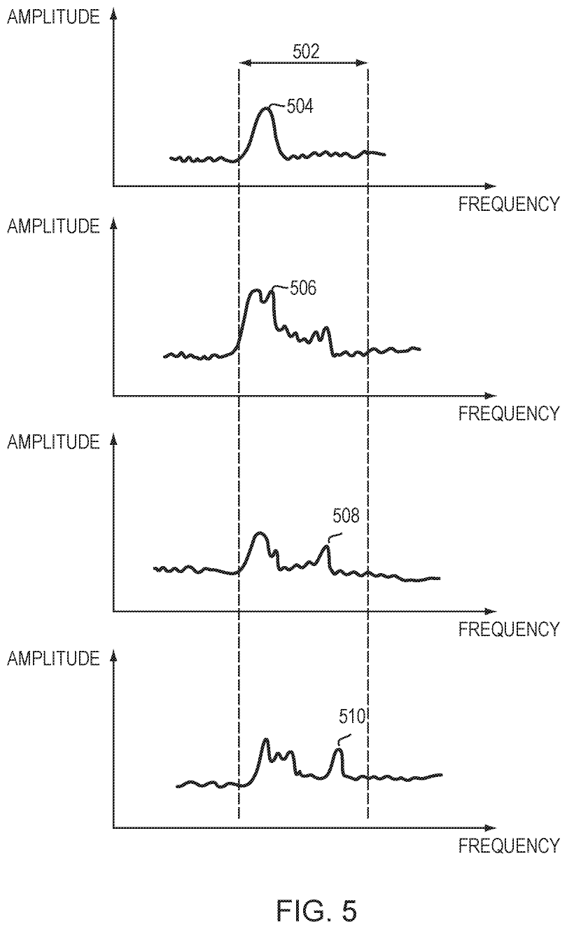

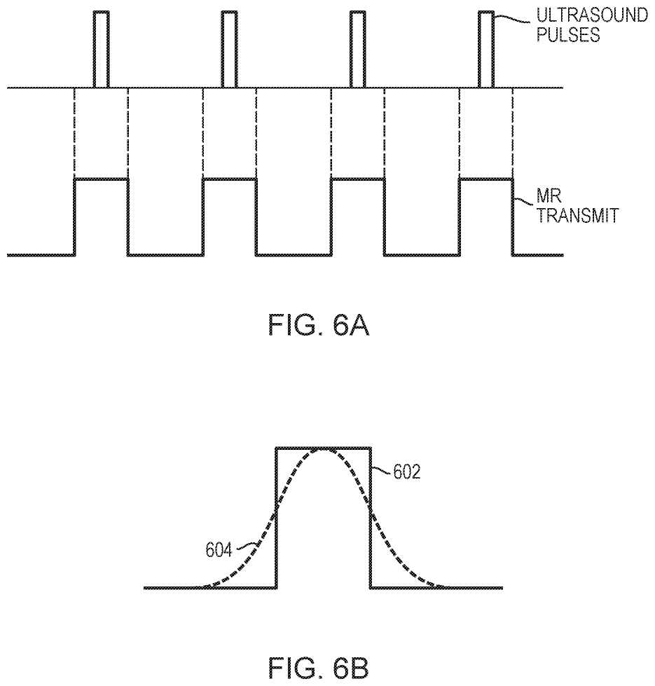

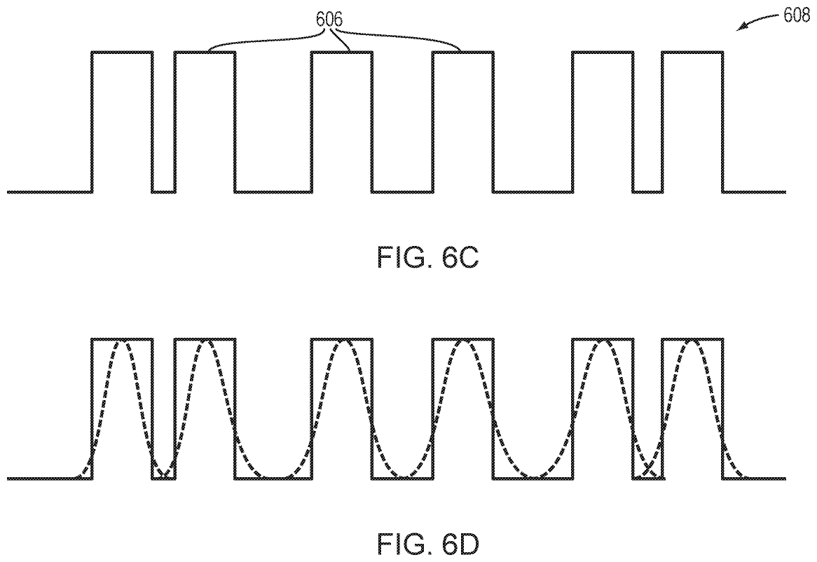

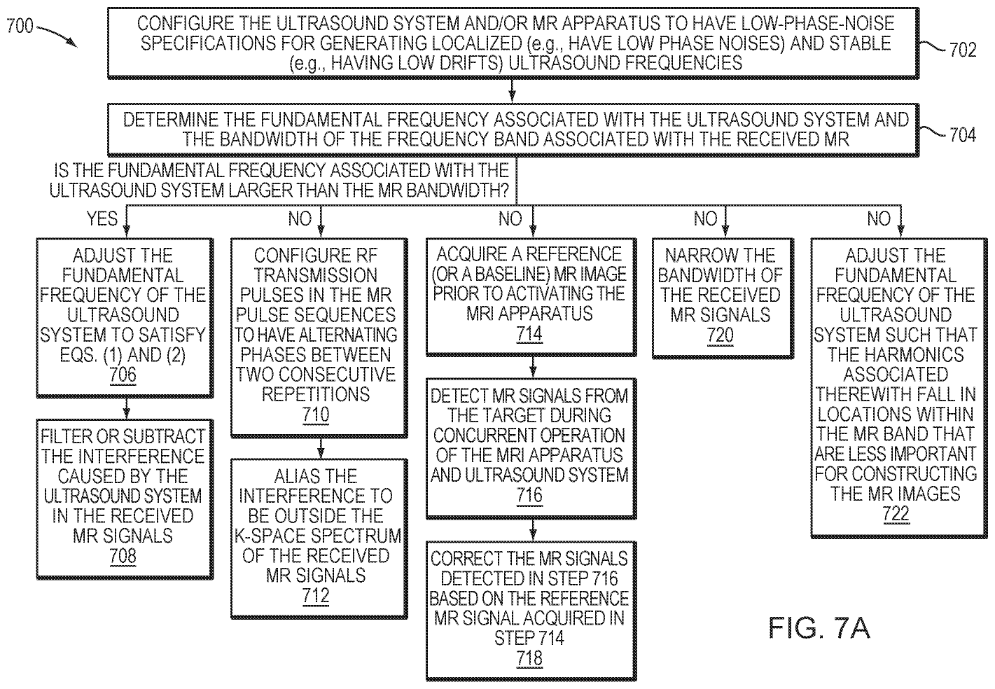

Referring to FIG. 6A, in various embodiments, the ultrasound system 202 is configured to operate on a pulsed (as opposed to continuous) basis. To avoid (or at least reduce) the interference between the ultrasound system 202 and the MRI apparatus 200, the ultrasound system 202 may be operated to transmit pulses only when the MRI apparatus is transmitting the MR pulse sequences, and deactivated while the MRI apparatus is receiving signals from the target. Approaches for operating the ultrasound system 202 based on the MRI apparatus are provided in, for example, U.S. Pat. No. 6,735,461 and U.S. Patent Publication No. 2016/0029969, the entire disclosures of which are hereby incorporated by reference.

Additionally or alternatively, the ultrasound pulses and/or the pulse envelope associated with the ultrasound fundamental frequency may be shaped so that the resulting fundamental frequency and harmonics form narrow bands and can thereby be easily filtered or subtracted. For example, referring to FIG. 6B, the ultrasound pulse 602 may be shaped to a new waveform 604 that has a relatively gradually-changing and smooth shape. In one embodiment, pulse-shaping is achieved using a suitable filter, such as a Gaussian filter, a raised-cosine filter, or a sinc filter. As a result, the fundamental frequency and corresponding harmonics associated with the new waveform 604 may form relatively narrow bands compared to those associated with the original pulse 602. The narrowed bands of fundamental frequency and harmonics may result in less interference with the MR received signals as well as easier filtering or subtraction from the received MR signals. Similarly, when the ultrasound pulse is sinusoidal, the envelope associated therewith may be shaped (e.g., by multiplying the pulse with a time window) to reduce the bandwidth of the fundamental frequency and/or harmonics.

Referring to FIG. 6C, in some embodiments, the controller 156 in the ultrasound system 202 may regulate the pulses 606 in a pulse train 608 such that the phase and/or time delay between some adjacent pulses 606 are different (or, in one embodiment, random). As a result, the noise associated with the fundamental frequency and harmonics may be stochastically spread over the spectrum in the frequency space and averaged out over application of the pulse train. This approach may effectively reduce the noise level caused by the ultrasound system 202 in the received MR signals. In addition, this approach may be combined with shaping of the ultrasound pulses (and/or the pulse envelope associated with the ultrasound fundamental frequency) described above (as depicted in FIG. 6D) so as to further reduce the interference between the ultrasound transducer and MRI apparatus.

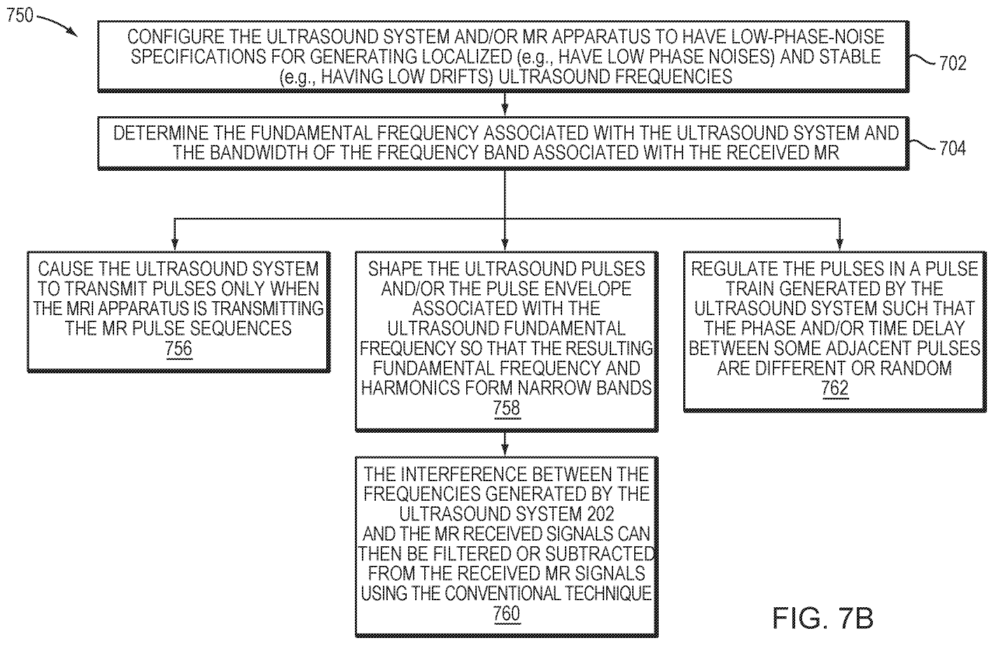

FIG. 7A depicts an exemplary approach 700 for eliminating (or at least reducing) interference between the frequencies of continuous waves generated by the ultrasound system 202 and the received MR signals in accordance herewith. In a first step 702, the ultrasound system 202 and/or MR apparatus 200 are configured to have low-phase-noise and/or low-frequency-drift specifications for generating localized (e.g., have low phase noises) and stable (e.g., having low drifts) ultrasound frequencies. For example, the ultrasound system 202 and/or MR apparatus 200 may employ a low-jitter (e.g., having a low phase noise) frequency generator and/or low-jitter switch elements (e.g., a switching amplifier). In addition, the oscillator implemented in the ultrasound system 202 and/or MR apparatus 200 may include a PLL and/or a DDS circuit to lock the frequency of the generated ultrasound signals to an MR internal clock of the MRI apparatus 200. In a second step 704, the fundamental frequency 302 associated with the ultrasound system 202 for optimizing diagnostic and/or therapeutic effects on the target as well as the frequency bandwidth of the received MR signals for optimizing MR imaging of the target are determined. If the fundamental frequency 302 associated with the ultrasound system 202 is larger than the bandwidth of the MR signals, the fundamental frequency of the ultrasound system is adjusted to satisfy Eqs. (1) and (2) set forth above (step 706). Thereafter, the interference caused by the ultrasound system in the received MR signals may be filtered or subtracted utilizing a suitable conventional technique (step 708). If, however, the fundamental frequency 302 is smaller than the MR bandwidth, the RF transmission pulses in the MR pulse sequences may be configured to have alternating phases between two consecutive repetitions (step 710). Subsequently, the interference between the ultrasound system and the MRI apparatus may be aliased outside the k-space spectrum of the received MR signals (step 712). Alternatively, a k-space or real-space reference (or a baseline) MR image resulting from operation of the ultrasound system 202 can be acquired prior to activating the MRI apparatus 202 (step 714). During concurrent operation of the MRI apparatus 200 and ultrasound system 202, the MR signals from the target may be detected (step 716) and then corrected by subtracting therefrom the reference signals measured when the MRI apparatus 200 is inactive or idling (step 718). In some embodiments, the bandwidth of the received MR signals is narrowed by, for example, increasing the MR sampling time and/or reducing the number of measured MR samples so as to reduce the interference with the ultrasound system 202 (step 720). Additionally or alternatively, the fundamental frequency of the ultrasound system 202 may be adjusted such that the harmonics associated therewith fall in locations within the MR band that are less important for constructing the MR images (step 722).

FIG. 7B depicts an exemplary approach 750 for eliminating (or at least reducing) interference between the frequencies of pulses generated by the ultrasound system 202 and the received MR signals in accordance herewith. Similar to approach 700 set forth in FIG. 7A, in a first step 702, the ultrasound system 202 and/or MR apparatus 200 are configured to have low-phase-noise and/or low-frequency-drift specifications for generating localized and stable ultrasound frequencies. Additionally, the fundamental frequency 302 associated with the ultrasound system 202 and the frequency bandwidth of the received MR signals may be determined (step 704). Thereafter, the ultrasound system 202 is operated to transmit pulses only when the MRI apparatus is transmitting the MR pulse sequences, and deactivated while the MRI apparatus is receiving signals from the target (step 756). Additionally or alternatively, the ultrasound pulses and/or the pulse envelope associated with the ultrasound fundamental frequency may be shaped using a suitable filter (such as a Gaussian filter, a raised-cosine filter, or a sinc filter) so that the resulting fundamental frequency and harmonics form narrow bands (step 758). The interference between the frequencies generated by the ultrasound system 202 and the MR received signals can then be filtered or subtracted from the received MR signals using a conventional technique (step 760). Additionally or alternatively, the pulses in a pulse train generated by the ultrasound system 202 are regulated such that the phase and/or time delay between some adjacent pulses are different or random (step 762). This may effectively reduce the noise level caused by the ultrasound system 202 in the received MR signals.

Accordingly, various embodiments first implement a frequency generator (and/or switch elements) having a low phase noise and/or a low frequency drift in the ultrasound system and/or MR apparatus to localize and stabilize the frequencies generated thereby. In addition, the generator may employ a PLL and/or DSS circuit to further stabilize the signals generated therefrom. Interference of the localized and stable signals generated by the ultrasound system and MRI apparatus may be more easily eliminated or reduced from the received MR signals using approaches 700, 750 described above.

In general, functionality for concurrently operating an MRI apparatus and an ultrasound system, including determining the fundamental frequency associated with the ultrasound system for optimizing diagnostic and/or therapeutic effects on the target, determining the bandwidth of the received MR signals associated with the MR apparatus for optimizing MR imaging of the target, adjusting the fundamental frequency generated by the ultrasound system, aliasing the interference between the ultrasound system and the MRI apparatus, adjusting the bandwidth of the received MR signals, filtering and/or subtracting the fundamental frequency and harmonics from the received MR signals, measuring reference MR signals, measuring MR signals during operation of the ultrasound system, generating a k-space or real-space MR image, shaping the pulses transmitted from the ultrasound system, and/or regulating the phase and/or time delay of the pulses transmitted from the ultrasound system, as described above, whether integrated with the controllers of MRI and/or the ultrasound system or provided by a separate external controller, may be structured in one or more modules implemented in hardware, software, or a combination of both. For embodiments in which the functions are provided as one or more software programs, the programs may be written in any of a number of high level languages such as PYTHON, FORTRAN, PASCAL, JAVA, C, C++, C #, BASIC, various scripting languages, and/or HTML. Additionally, the software can be implemented in an assembly language directed to the microprocessor resident on a target computer (e.g., the controller); for example, the software may be implemented in Intel 80×86 assembly language if it is configured to run on an IBM PC or PC clone. The software may be embodied on an article of manufacture including, but not limited to, a floppy disk, a jump drive, a hard disk, an optical disk, a magnetic tape, a PROM, an EPROM, EEPROM, field-programmable gate array, or CD-ROM. Embodiments using hardware circuitry may be implemented using, for example, one or more FPGA, CPLD or ASIC processors.

In addition, the term “controller” used herein broadly includes all necessary hardware components and/or software modules utilized to perform any functionality as described above; the controller may include multiple hardware components and/or software modules and the functionality can be spread among different components and/or modules. Further, the MRI controller 116 may be separate from the ultrasound controller 156 or may be combined with the ultrasound controller 156 into an integrated system control facility.

Certain embodiments of the present invention are described above. It is, however, expressly noted that the present invention is not limited to those embodiments; rather, additions and modifications to what is expressly described herein are also included within the scope of the invention.

What is claimed is:

1. A system for performing magnetic resonance (MR) imaging of an anatomic region in conjunction with an ultrasound operation on the anatomic region, the system comprising:
an MR imaging apparatus for imaging the anatomic region;
an ultrasound transducer system for performing the ultrasound operation; and
a controller in communication with the MR imaging apparatus and ultrasound transducer system, the controller being configured to:
cause the ultrasound transducer system to transmit, to the anatomic region, ultrasound waves or pulses having a fundamental frequency and a plurality of harmonics;
cause the MR imaging apparatus to transmit an MR pulse sequence to the anatomic region and receive, therefrom, MR signals within a band of frequencies; and
cause the band of the frequencies to be located between two adjacent frequencies of the harmonics.

2. The system of claim 1, where in the ultrasound transducer system comprises at least one of a low-jitter frequency generator or a low-jitter switch element for reducing a phase noise associated with the fundamental frequency and harmonics.

3. The system of claim 1, wherein at least one of the ultrasound transducer system or the MR imaging apparatus comprises at least one oscillator having a low frequency drift so as to improve stability of the fundamental frequency, the harmonics and/or a frequency associated with ultrasound waves or pulses transmitted by the MR imaging apparatus.

4. The system of claim 3, wherein the at least one oscillator comprises a phase-locked loop for locking a phase associated with the fundamental frequency, the harmonics and/or the frequency associated with the ultrasound waves or pulses transmitted by the MR imaging apparatus to an internal clock of the MR imaging apparatus.

5. The system of claim 1, wherein the controller is further configured to filter or subtract the fundamental frequency and harmonics from the received MR signals.

6. The system of claim 1, wherein the fundamental frequency is larger than a bandwidth of the received MR signals.

7. The system of claim 1, wherein the MR pulse sequence comprises RF transmission pulses having alternating phases between two consecutive repetitions.

8. The system of claim 1, wherein the controller is further configured to:
cause the MR imaging apparatus to detect reference MR signals in response to transmission of the ultrasound waves or pulses thereto prior to causing the MR imaging apparatus to transmit the MR pulse sequence to the anatomic region; and
adjust the received MR signals based at least in part on the reference MR signals.

9. The system of claim 1, wherein the controller is further configured to reduce a bandwidth of the received MR signals.

10. The system of claim 9, wherein the controller is further configured to increase an MR scanning time or reduce a number of measured MR signals.

11. The system of claim 1, wherein the controller is further configured to shape a waveform of at least one of the ultrasound pulses.

12. The system of claim 9, wherein the controller is further configured to implement at least one of a Gaussian filter, a raised-cosine filter, or a sinc filter for shaping the waveform of said at least one of the ultrasound pulses.

13. The system of claim 1, wherein the controller is further configured to regulate the ultrasound pulses such that a phase and/or a time delay between some of the pulses are different.

14. The system of claim 1, wherein the controller is implemented in the ultrasound transducer system.

15. A method of performing magnetic resonance (MR) imaging of an anatomic region in conjunction with an ultrasound operation on the anatomic region, the method comprising:
transmitting a plurality of ultrasound waves or pulses having a fundamental frequency and a plurality of harmonics to the anatomic region;
transmitting an MR pulse sequence to the anatomic region and receiving, therefrom, MR signals within a band of frequencies; and
causing the band of frequencies to be located between two adjacent frequencies of the harmonics.

16. The method of claim 15, further comprising filtering or subtracting the fundamental frequency and harmonics from the received MR signals.

17. The method of claim 15, wherein the fundamental frequency is larger than a bandwidth of the received MR signals.

18. The method of claim 15, wherein the MR pulse sequence comprises RF transmission pulses having alternating phases between two consecutive repetitions.

19. The method of claim 15, further comprising:
causing the MR imaging apparatus to detect reference MR signals in response to transmission of the ultrasound waves or pulses thereto prior to causing the MR imaging apparatus to transmit the MR pulse sequence to the anatomic region; and
adjusting the received MR signals based at least in part on the reference MR signals.

20. The method of claim 15, further comprising reducing a bandwidth of the received MR signals.

21. The method of claim 20, further comprising increasing an MR scanning time or reducing a number of measured MR signals.

22. The method of claim 15, further comprising shaping a waveform of at least one of the ultrasound pulses.

23. The method of claim 22, wherein the waveform of said at least one of the ultrasound pulses is shaped by at least one of a Gaussian filter, a raised-cosine filter, or a sinc filter.

24. The method of claim 15, further comprising regulating the ultrasound pulses such that a phase and/or a time delay between some of the pulses are different.

25. A system for performing magnetic resonance (MR) imaging of an anatomic region in conjunction with an ultrasound operation on the anatomic region, the system comprising:
an MR imaging apparatus for imaging the anatomic region;
an ultrasound transducer system for performing the ultrasound operation; and
a controller in communication with the MR imaging apparatus and ultrasound transducer system, the controller being configured to:
cause the ultrasound transducer system to transmit, to the anatomic region, ultrasound waves or pulses having a fundamental frequency and a plurality of harmonics; and
cause the MR imaging apparatus to transmit an MR pulse sequence having a plurality of RF transmission pulses to the anatomic region and receive, therefrom, MR signals within a band of frequencies,
wherein the RF transmission pulses have alternating phases between two consecutive repetitions.

26. The system of claim 25, where in the ultrasound transducer system comprises at least one of a low-jitter frequency generator or a low-jitter switch element for reducing a phase noise associated with the fundamental frequency and harmonics.

27. The system of claim 25, wherein at least one of the ultrasound transducer system or the MR imaging apparatus comprises at least one oscillator having a low frequency drift so as to improve stability of the fundamental frequency, the harmonics and/or a frequency associated with ultrasound waves or pulses transmitted by the MR imaging apparatus.

28. The system of claim 27, wherein the at least one oscillator comprises a phase-locked loop for locking a phase associated with the fundamental frequency, the harmonics and/or the frequency associated with the ultrasound waves or pulses transmitted by the MR imaging apparatus to an internal clock of the MR imaging apparatus.

29. The system of claim 25, wherein the controller is further configured to filter or subtract the fundamental frequency and harmonics from the received MR signals.

30. The system of claim 25, wherein the fundamental frequency is smaller than a bandwidth of the received MR signals.

31. The system of claim 25, wherein the controller is further configured to:
cause the MR imaging apparatus to detect reference MR signals in response to transmission of the ultrasound waves or pulses thereto prior to causing the MR imaging apparatus to transmit the MR pulse sequence to the anatomic region; and
adjust the received MR signals based at least in part on the reference MR signals.

32. The system of claim 25, wherein the controller is further configured to reduce a bandwidth of the received MR signals.

33. The system of claim 32, wherein the controller is further configured to increase an MR scanning time or reduce a number of measured MR signals.

34. The system of claim 25, wherein the controller is further configured to shape a waveform of at least one of the ultrasound pulses.

35. The system of claim 34, wherein the controller is configured to implement at least one of a Gaussian filter, a raised-cosine filter, or a sinc filter for shaping the waveform of said at least one of the ultrasound pulses.

36. The system of claim 25, wherein the controller is further configured to regulate the ultrasound pulses such that a phase and/or a time delay between some of the pulses are different.

37. The system of claim 25, wherein the controller is implemented in the ultrasound transducer system.

38. A method of performing magnetic resonance (MR) imaging of an anatomic region in conjunction with an ultrasound operation on the anatomic region, the method comprising:
transmitting a plurality of ultrasound waves or pulses having a fundamental frequency and a plurality of harmonics to the anatomic region; and
transmitting an MR pulse sequence having a plurality of RF transmission pulses to the anatomic region and receiving, therefrom, MR signals within a band of frequencies,
wherein the RF transmission pulses have alternating phases between two consecutive repetitions.

39. The method of claim 38, further comprising filtering or subtracting the fundamental frequency and harmonics from the received MR signals.

40. The method of claim 38, wherein the fundamental frequency is smaller than a bandwidth of the received MR signals.

41. The method of claim 38, further comprising:
causing the MR imaging apparatus to detect reference MR signals in response to transmission of the ultrasound waves or pulses thereto prior to causing the MR imaging apparatus to transmit the MR pulse sequence to the anatomic region; and
adjusting the received MR signals based at least in part on the reference MR signals.

42. The method of claim 38, further comprising reducing a bandwidth of the received MR signals.

43. The method of claim 42, further comprising increasing an MR scanning time or reducing a number of measured MR signals.

44. The method of claim 38, further comprising shaping a waveform of at least one of the ultrasound pulses.

45. The method of claim 44, wherein the waveform of said at least one of the ultrasound pulses is shaped by at least one of a Gaussian filter, a raised-cosine filter, or a sinc filter.

46. The method of claim 38, further comprising regulating the ultrasound pulses such that a phase and/or a time delay between some of the pulses are different.
